(19) United States
(12) Patent Application Publication
(10) Pub. No.: US 2012/0238782 A1
(43) Pub. Date: Sep. 20, 2012

(54) METHODS FOR PRODUCING ALLYL CHLORIDE AND DICHLOROHYDRIN

(76) Inventors: Masahiro Kinoshita, Niihama-shi (JP); Norihito Omoto, Niihama-shi (JP)

(21) Appl. No.: 13/510,259

(22) PCT Filed: Oct. 20, 2010
(86) PCT No.: PCT/JP2010/006215

§ 371 Date: May 16, 2012

(30) Foreign Application Priority Data
Nov. 17, 2009 (JP) 2009-261591

(51) Int. Cl. C07C 17/02; C07C 41/06

(52) U.S. Cl. 568/681; 570/223

(57) ABSTRACT

A method for producing allyl chloride is provided which includes: a chlorination step of reacting propylene with chlorine to obtain a reaction product containing allyl chloride, unreacted propylene and by-product hydrogen chloride; a separation step of cooling the reaction product to separate the reaction product into the allyl chloride and a mixed gas containing the unreacted propylene and the by-product hydrogen chloride; a collection step of separating the mixed gas into the unreacted propylene and the by-product hydrogen chloride to collect the unreacted propylene; and an oxidation step of oxidizing the separated by-product hydrogen chloride to obtain chlorine. At least part of the chlorine used in the chlorination step is the chlorine obtained in the oxidation step. A method for producing dichlorohydrin using the chlorine obtained in the oxidation step is also provided.

### TECHNICAL FIELD

The present invention relates to methods for producing allyl chloride and dichlorohydrin, and more specifically, to methods for producing allyl chloride and dichlorohydrin in which hydrogen chloride obtained as a by-product in producing allyl chloride is effectively utilized as a raw material to be reused.

### BACKGROUND ART

A method for producing allyl chloride by the gas-phase reaction of propylene with chlorine is widely used as an industrial production method of allyl chloride (for example, see Patent Document 1). In this production process, hydrogen chloride (HCl) equimolar to allyl chloride is obtained as a by-product, as represented in the following expression.

C₃H₆+Cl₂→C₃H₅Cl+HCl [Math. 1]

In the industrial production of allyl chloride, particularly, in the continuous industrial production thereof, it is desirable that hydrogen chloride thus produced in large quantity is stably disposed of while being effectively utilized, concurrently with production of allyl chloride. The method of processing hydrogen chloride obtained as a by-product may include reusing it as a raw material for producing other products such as vinyl chloride, and utilizing it as neutralizer for alkaline liquid (such as alkaline waste liquid) generated in a production process of various products. However, since the consumption amount of by-product hydrogen chloride in each of these processing methods is dependent on the balance of supply and demand of the products by consumers, each of the methods cannot necessarily be a method capable of stably disposing of by-product hydrogen chloride.

### CITATION LIST

### Patent Literature

PTL 1: Japanese Patent Laying-Open No. 60-252434

### SUMMARY OF INVENTION

### Technical Problem

Thus, an object of the present invention is to provide a method capable of stably disposing of and effectively utilizing by-product hydrogen chloride obtained in the production process of allyl chloride by the reaction of propylene with chlorine, and particularly, in the continuous industrial production process thereof.

### Solution to Problem

In order to solve the above-described problems, the present invention provides a method for producing allyl chloride, the method including the following steps: a chlorination step of reacting propylene with chlorine to obtain a reaction product containing allyl chloride, unreacted propylene and by-product hydrogen chloride; a separation step of cooling the reaction product to separate the reaction product into the allyl chloride and a mixed gas containing the unreacted propylene and the by-product hydrogen chloride; a collection step of separating the mixed gas into the unreacted propylene and the by-product hydrogen chloride to collect the unreacted propylene; and an oxidation step of oxidizing the by-product hydrogen chloride separated in the collection step to obtain chlorine. In the present invention, at least part of the chlorine used in the chlorination step is the chlorine obtained in the oxidation step.

It is preferable that the method for producing allyl chloride according to the present invention further includes, between the collection step and the oxidation step, an impurity removal step of removing an impurity from the by-product hydrogen chloride separated in the collection step. The impurity removed in the impurity removal step preferably includes at least a bromine containing component. In the impurity removal step, in addition to the bromine containing component, one or more types of organic impurities selected from the group consisting of unreacted propylene, isopropanol, 2-chloropropane and allyl chloride may be further removed.

The oxidation step preferably includes the step of oxidizing the by-product hydrogen chloride with oxygen gas in presence of a catalyst. Furthermore, the collection step preferably includes the step of contacting the mixed gas with water to separate the by-product hydrogen chloride as hydrochloric acid.

Furthermore, the present invention provides a method for producing dichlorohydrin by a reaction of allyl chloride, chlorine and water, in which at least part of the chlorine is the chlorine obtained in the oxidation step included in the above-described method for producing allyl chloride. It is preferable that at least part of the allyl chloride used in the method for producing dichlorohydrin according to the present invention is the allyl chloride obtained in the separation step included in the above-described method for producing allyl chloride.

### Advantageous Effects of Invention

According to the method for producing allyl chloride and the method for producing dichlorohydrin in accordance with the present invention, by-product hydrogen chloride obtained in the production process of allyl chloride by the reaction of propylene with chlorine is oxidized to chlorine and then reused as a material for producing allyl chloride or dichlorohydrin, thereby allowing the by-product hydrogen chloride to be stably disposed of while being effectively utilized.

### BRIEF DESCRIPTION OF DRAWINGS

FIG. 1 is a flowchart schematically showing an example of a method for producing allyl chloride according to the present invention.

FIG. 2 is a flowchart schematically showing an example of a method for producing dichlorohydrin according to the present invention.

### DESCRIPTION OF EMBODIMENTS

<Method for Producing Allyl Chloride>

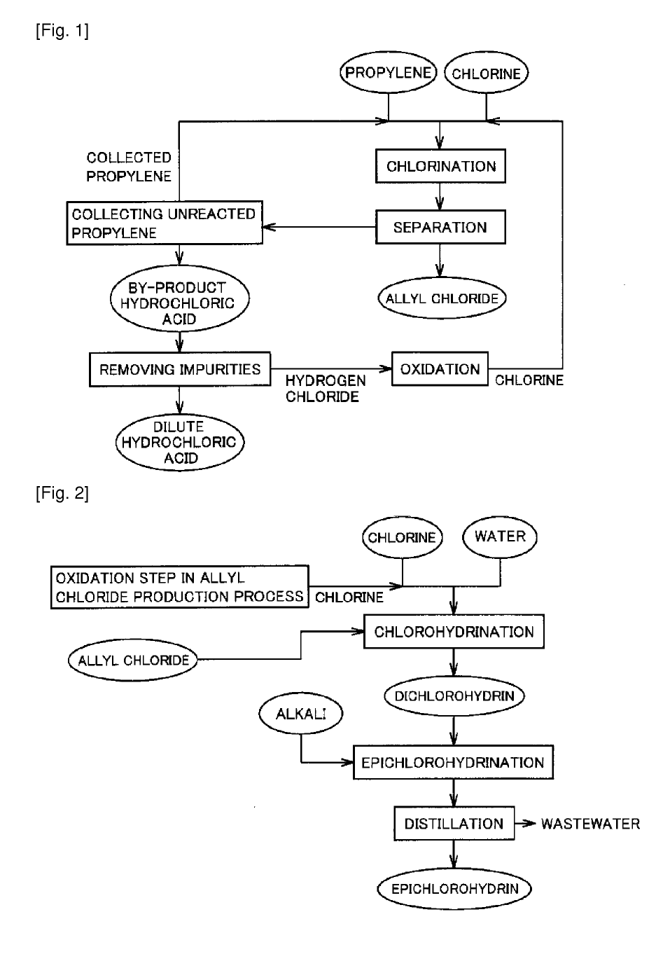

FIG. 1 is a flowchart schematically showing an example of a method for producing allyl chloride according to the present invention. As shown in FIG. 1, the method for producing allyl chloride according to the present invention basically includes the following steps:

(1) a chlorination step of reacting propylene with chlorine to obtain a reaction product containing allyl chloride, unreacted propylene and by-product hydrogen chloride;

(2) a separation step of cooling the reaction product to separate the reaction product into the allyl chloride and a mixed gas containing the unreacted propylene and the by-product hydrogen chloride;

(3) a collection step of separating the mixed gas into the unreacted propylene and the by-product hydrogen chloride to collect the unreacted propylene;

(4) an impurity removal step of removing an impurity from the by-product hydrogen chloride separated in the collection step; and

(5) an oxidation step of oxidizing the by-product hydrogen chloride having the impurity removed therefrom, to obtain chlorine.

It is to be noted that the above-described impurity removal step is optional, and chlorine may be obtained by directly oxidizing the by-product hydrogen chloride separated in the collection step. Each of the steps will be hereinafter described in detail.

(1) Chlorination Step

This step includes a gas-phase reaction of propylene with chlorine to obtain a gaseous reaction product mainly containing allyl chloride, unreacted propylene and by-product hydrogen chloride. The gas-phase reaction may be a continuous reaction in which propylene gas and chlorine gas are continuously supplied to a chlorination reactor. The reaction temperature is usually approximately 450 to 510° C. In the present invention, chlorine obtained in the oxidation step which will be described later is used as at least part of the chlorine used in the reaction with propylene. Consequently, by-product hydrogen chloride obtained by the above-described gas-phase reaction can be effectively utilized and can be stably disposed of.

The gaseous reaction product obtained by the reaction usually contains impurities generated by the side reaction such as poly-chlorinated propylenes and 2-chloropropane, in addition to allyl chloride, unreacted propylene and by-product hydrogen chloride. Furthermore, while electrolysis of salt such as rock salt and sea salt or potassium chloride is widely used as an industrial production process of chlorine. Chlorine obtained by this process contains bromine (Br₂) of several ten ppm to several hundred ppm. Accordingly, in the case where chlorine produced by the above-described electrolysis is used as chlorine used for the reaction with propylene, the gaseous reaction product contains a bromine containing component such as brominated propylenes and hydrogen bromide as impurities.

(2) Separation Step

The above-described gaseous reaction product is then cooled and separated into condensed allyl chloride and a mixed gas containing unreacted propylene and by-product hydrogen chloride. The cooling temperature is approximately 50 to 70° C., for example. The cooling method may include, for example, introducing the reaction product into a heat exchanger for cooling, introducing the reaction product into quenching equipment such as a line quencher for cooling, employing both of these methods, or the like. Particularly, in terms of cooling efficiency and separation efficiency, it is preferable to employ a method of pre-cooling the reaction product using a heat exchanger and thereafter introducing the same into quenching equipment such as a line quencher for rapid quenching. The pre-cooling temperature can be set at 150 to 250° C., for example.

The condensed phase obtained by cooling, which consists mainly of allyl chloride, may contain high-boiling impurities such as poly-chlorinated propylenes and brominated propylenes. Accordingly, a purification process such as distillation may be performed optionally, in order to ensure the purity for a product of allyl chloride or the purity for a material (for example, material used for producing dichlorohydrin which will be described later).

The gas phase obtained by the cooling process consists of a mixed gas mainly containing unreacted propylene and by-product hydrogen chloride. The gas phase may contain impurities such as allyl chloride, poly-chlorinated propylenes, brominated propylenes, 2-chloropropane and hydrogen bromide. However, most of the poly-chlorinated propylenes and the brominated propylenes are distributed to the condensed phase.

The process of collecting allyl chloride by cooling as described above may be performed more than once. Furthermore, prior to a collection step of collecting unreacted propylene, the gas phase obtained by the cooling process may be distilled under high pressure, for example, to collect allyl chloride contained in the gas phase.

(3) Collection Step

Then, the mixed gas separated in the above-described separation step and mainly containing unreacted propylene and by-product hydrogen chloride is separated into the unreacted propylene and the by-product hydrogen chloride. The separated and collected unreacted propylene can be reused as a raw material for the above-described chlorination reaction (see FIG. 1).

The method of separating the mixed gas into the unreacted propylene and the by-product hydrogen chloride may include contacting the mixed gas with water to cause the by-product hydrogen chloride contained in the mixed gas to be absorbed in water, thereby separating the by-product hydrogen chloride as hydrochloric acid (which will be hereinafter also referred to as by-product hydrochloric acid) and collecting the unreacted propylene as unabsorbed gas, as shown in FIG. 1. In this method, in place of water, dilute hydrochloric acid can be brought into contact with the mixed gas. Water or dilute hydrochloric acid can be brought into contact with the mixed gas, for example, using an absorption tower. In this case, the unreacted propylene is collected through the top of the absorption tower while the by-product hydrochloric acid is obtained as a bottom product.

The hydrogen chloride concentration of the by-product hydrochloric acid obtained by absorption of the by-product hydrogen chloride into water or dilute hydrochloric acid is not particularly limited, but can be set at approximately 25 to 37 percent by mass, for example. The by-product hydrochloric acid may contain impurities such as unreacted propylene, allyl chloride, poly-chlorinated propylenes, brominated propylenes, isopropanol, 2-chloropropane, hydrogen bromide, and the like.

It is to be noted that the mixed gas can be separated into the unreacted propylene and the by-product hydrogen chloride also by distilling the mixed gas under high pressure.

(4) Impurity Removal Step

This step is to remove an impurity from the by-product hydrogen chloride or the by-product hydrochloric acid separated in the above-described collection step. The impurity removal step is not indispensable but is preferable to be provided in the present invention for the following reasons. When chlorine produced by electrolysis of salt or potassium chloride is used as a raw material for producing allyl chloride, the bromination reaction caused by bromine contained in the chlorine occurs as a side reaction, as described above. This causes generation of impurities such as brominated propylenes, thereby leading to a decrease in yield of allyl chloride. Furthermore, the reaction of bromine with propylene generates hydrogen bromide as well as brominated propylene. The hydrogen bromide may be accompanied by the above-described by-product hydrochloric acid and converted into bromine in the oxidation step described below, and may be resupplied in the chlorination step together with the chlorine to be reused. Such an unintentional recycling of bromine also leads to generation of impurities and a decrease in yield of allyl chloride. Similarly, chlorine obtained in the oxidation step is used as a raw material for dichlorohydrin, which may also lead to generation of impurities and a decrease in yield of dichlorohydrin.

When the impurity removal step of removing at least a bromine containing component such as brominated propylenes and hydrogen bromide is provided between the collection step and the oxidation step, chlorine can be obtained that, in the oxidation step, does not contain bromine or contains an extremely small content of bromine as compared with chlorine produced by electrolysis. Therefore, when chlorine obtained through the impurity removal step is used as a raw material for producing allyl chloride or dichlorohydrin, generation of the bromine containing impurity can be suppressed and yield thereof can be improved.

The method of removing the bromine containing component such as brominated propylenes and hydrogen bromide may include the method disclosed in Japanese Patent Laying-Open No. 2009-001459. Specifically, the method includes the first step of introducing the by-product hydrochloric acid into a first separation tower, and distilling or stripping the same with inert gas, thereby removing most of organic impurities such as brominated propylenes as distillate gas and obtaining a first hydrochloric acid as a bottom product; and the second step of distilling the first hydrochloric acid in a second separation tower, thereby obtaining, through the top of the tower, a highly purified hydrogen chloride gas as distillate gas from which at least the bromine containing component is removed. According to this method, the impurity hydrogen bromide can be removed from the by-product hydrochloric acid since it remains in a dilute hydrochloric acid that is a bottom product resulting from distillation in the second separation tower.

The distillation pressure in the first step is not particularly limited, but can be set approximately at atmospheric pressure (0 MPaG, gauge pressure). The distillation temperature is preferably set at a relatively lower temperature in order to distribute the organic impurities such as brominated propylene selectively to the tower top side. The temperature at the tower top is preferably set at approximately 10 to 90° C. when the distillation pressure is atmospheric pressure. The temperature within the tower is set as appropriate to reach the above-described preferable temperature at the tower top, and can be set at approximately 30 to 100° C. when the distillation pressure is atmospheric pressure.

In the case where stripping is performed with inert gas in the first step, the pressure during the stripping is typically set approximately at atmospheric pressure. The temperature within the tower during the stripping is typically set at approximately 20 to 50° C. The specific method of stripping with inert gas may include blowing the inert gas from below into a shower of the by-product hydrochloric acid. The inert gas may be, but not particularly limited to, for example, nitrogen gas, air, helium gas, argon gas, or the like. The supply amount of inert gas (kg) is preferably set at proximately 0.1 to 0.5 with respect to the by-product hydrochloric acid (kg) supplied into the first separation tower.

The first hydrochloric acid obtained as a bottom product by distillation or stripping in the first separation tower is discharged from the bottom of the first separation tower, supplied into the second separation tower and subjected to the second step.

The distillation pressure in the second step is not particularly limited. However, when the distillate gas discharged from the top of the tower is directly introduced into the subsequent oxidation step, it is preferable that the distillation pressure is set at approximately 0.1 to 1 MPaG (gauge pressure) since the oxidation step is typically performed at approximately 0.1 to 1 MPaG. The distillation pressure is more preferably set at approximately 0.1 to 0.7 MPaG. The temperature within the tower reaches the temperature corresponding to the distillation pressure, which is typically at approximately 100 to 180° C. Reflux at the top of the tower allows hydrogen chloride gas of much higher purity to be produced.

As disclosed in Japanese Patent Laying-Open No. 2009-001459, in order to improve the collection rate of hydrogen chloride in the impurity removal step, the distillate gas collected from the top of the tower in the first step and/or the bottom product in the second step may be recycled.

In the present step, at least bromine containing component such as brominated propylenes and hydrogen bromide are preferably removed as described above. It is also preferable that impurities other than the bromine containing component are also removed for the purpose of maintaining the activity of the catalyst used in the oxidation step and reducing impurities. The impurities other than the bromine containing component may include organic impurities such as unreacted propylene, allyl chloride, poly-chlorinated propylenes, isopropanol and 2-chloropropane. Preferably, at least one type or two or more types of these organic impurities are removed together with the bromine containing component. More preferably, all of these organic impurities are removed together with the bromine containing component. According to the method including the first and second steps as described above, these organic impurities can also be usually removed together with the bromine containing component.

(5) Oxidation Step

Then, the by-product hydrogen chloride is oxidized to obtain chlorine in the present step. The by-product hydrogen chloride used herein refers to resultant by-product hydrochloric acid or hydrogen chloride collected therefrom by distillation or the like in the case where unreacted propylene is collected in the collection step by absorption into water or the like, and refers to hydrogen chloride obtained by high-pressure distillation in the case where unreacted propylene is collected in the collection step by the distillation. Furthermore, the by-product hydrogen chloride used herein refers to distillate gas consisting of highly purified hydrogen chloride gas obtained in the second step in the case where the method is employed that is provided with the impurity removal step including the above-described first and second steps.

The method of oxidizing the by-product hydrogen chloride may include, for example, 1) electrolysis of the by-product hydrochloric acid and 2) oxidizing, in the presence of an oxidation catalyst such as ruthenium oxide, the by-product hydrochloric acid, hydrogen chloride collected therefrom by distillation or the like, or the distillate gas obtained through the impurity removal step. In particular, the latter method by catalyst oxidation is excellent in energy efficiency since it allows heat recovery such as by generating steam with recovered reaction heat. In addition, the method is also advantageous in that it does not require hydrogen chloride of high purity as compared with the method by electrolysis. In contrast, since the method by electrolysis requires large power consumption and causes generation of hydrogen which is problematic in terms of safety, the method tends to be less advantageous as compared with the method by catalyst oxidation, particularly from an industrial viewpoint. Furthermore, since the electrolysis is interfered with a small amount of organic substances in the hydrochloric acid, hydrochloric acid of extremely high purity is required.

As the specific method of catalyst oxidation, the conventionally known method can be employed. Preferably, the method disclosed in Japanese Patent Laying-Open No. 2009-001459 can be employed, for example. This document discloses the process for producing chlorine including: an oxidation step of oxidizing hydrogen chloride gas with oxygen to obtain a gas containing chlorine; an absorption step of contacting the gas containing chlorine obtained in the oxidation step with water or hydrochloric acid to collect unreacted hydrogen chloride as a solution containing hydrogen chloride and water as main components and to obtain a gas containing chlorine and unreacted oxygen as main components; a drying step of drying the gas obtained in the absorption step; and a purification step of separating the gas obtained in the drying step into a solution or a gas containing chlorine as a main component and a gas containing unreacted oxygen as a main component.

The chlorine obtained in the present step is recycled to the above-described chlorination step as a raw material for the chlorination reaction of propylene. According to the method for producing allyl chloride in accordance with the present invention as described above, hydrogen chloride obtained as a by-product can be effectively utilized and the by-product hydrogen chloride can also be stably disposed of. Furthermore, when chlorine having a bromine containing component removed therefrom is recycled, generation of impurities derived from the bromine containing component can be suppressed, which allows improvement in purity and yield of allyl chloride.

<Method for Producing Dichlorohydrin>

FIG. 2 is a flowchart schematically showing an example of the method for producing dichlorohydrin according to the present invention. FIG. 2 also shows a flow in which the obtained dichlorohydrin is used to produce epichlorohydrin. In the method for producing dichlorohydrin according to the present invention, chlorine obtained in the oxidation step in the method for producing allyl chloride according to the present invention as described above is used as at least part of chlorine which is a raw material, in the method for producing dichlorohydrin by the reaction of allyl chloride, chlorine and water as represented in the following expression:

C₃H₅Cl+HOCl→C₃H₆OCl₂ [Math.2]

In this way, by-product hydrogen chloride can be stably disposed of while being effectively utilized also by using, as a raw material for producing dichlorohydrin, chlorine derived from the by-product hydrogen chloride obtained by the method for producing allyl chloride according to the present invention as described above. For the purpose of reducing generation of impurities in chlorohydrination and improving yield of dichlorohydrin, the chlorine supplied from the allyl chloride production process is preferably the chlorine produced through the above-described impurity removal step.

Furthermore, in the method for producing dichlorohydrin according to the present invention, allyl chloride obtained by the method for producing allyl chloride according to the present invention, that is, allyl chloride obtained through the chlorination step and the separation step may be used as at least part of allyl chloride which is used as a raw material. Prior to chlorohydrination, the allyl chloride may be optionally subjected to the purification process (distillation and the like) for removal of impurities and the like.

The method for producing dichlorohydrin by the reaction of allyl chloride, chlorine and water may include, for example, reacting a mixture that contains equimolar amounts of allyl chloride and chlorine and also contains water as a solvent at a temperature of 10 to 60° C. This results in an aqueous solution containing dichlorohydrin.

The obtained dichlorohydrin can be suitably used as a raw material for producing epichlorohydrin (see FIG. 2). Epichlorohydrin can be produced by the method in which the above-described dichlorohydrin aqueous solution is reacted with alkali, and the obtained epichlorohydrin aqueous solution is then distilled to collect epichlorohydrin as an overhead component. The examples of the alkali may include hydroxide and carbonate of alkali metals or alkaline-earth metals, such as sodium hydroxide, calcium hydroxide and calcium carbonate.

1. A method for producing allyl chloride, comprising the steps of:
a chlorination step of reacting propylene with chlorine to obtain a reaction product containing allyl chloride, unreacted propylene and by-product hydrogen chloride;
a separation step of cooling the reaction product to separate the reaction product into the allyl chloride and a mixed gas containing the unreacted propylene and the by-product hydrogen chloride;
a collection step of separating the mixed gas into the unreacted propylene and the by-product hydrogen chloride, to correct the unreacted propylene; and
an oxidation step of oxidizing the by-product hydrogen chloride separated in the collection step to obtain chlorine,
at least part of the chlorine used in the chlorination step being the chlorine obtained in the oxidation step.

2. The method for producing allyl chloride according to claim 1, further comprising, between the collection step and the oxidation step, the step of removing an impurity from the by-product hydrogen chloride separated in the collection step.

3. The method for producing allyl chloride according to claim 2, wherein the impurity includes at least a bromine containing component.

4. The method for producing allyl chloride according to claim 3, wherein the impurity further includes one or more types of organic impurities selected from the group consisting of unreacted propylene, isopropanol, 2-chloropropane and allyl chloride.

5. The method for producing allyl chloride according to claim 1, wherein the oxidation step includes the step of oxidizing the by-product hydrogen chloride with oxygen gas in presence of a catalyst.

6. The method for producing allyl chloride according to claim 1, wherein the collection step includes the step of contacting the mixed gas with water to separate the by-product hydrogen chloride as hydrochloric acid.

7. A method for producing dichlorohydrin by a reaction of allyl chloride, chlorine and water,
at least part of the chlorine being the chlorine obtained in the oxidation step according to claim 1.

8. A method for producing dichlorohydrin by a reaction of allyl chloride, chlorine and water,
at least part of the chlorine being the chlorine obtained in the oxidation step according to claim 1, and
at least part of the allyl chloride being the allyl chloride obtained in the separation step according to claim 1.
